(19) United States
(12) Patent Application Publication
(10) Pub. No.: US 2025/0187754 A1
(43) Pub. Date: Jun. 12, 2025

(54) Pre-Flight Caddy

(71) Applicant: Board of Trustees of Western Michigan University, Kalamazoo, MI (US)

(72) Inventors: Eric David Effinger, Greenfield, WI (US); Matilda McLean, Portage, MI (US); William Feenstra, Battle Creek, MI (US); Scott Austin, Battle Creek, MI (US)

(21) Appl. No.: 18/973,597

(22) Filed: Dec. 9, 2024

Related U.S. Application Data
(60) Provisional application No. 63/607,824, filed on Dec. 8, 2023.

(51) Int. Cl. B64F 1/28; B64F 5/60; G01N 33/28

(52) U.S. Cl. CPC B64F 1/28; B64F 5/60; G01N 33/2829

(57) ABSTRACT

Some embodiments of the present invention may include a caddy for securely storing aircraft pre-flight fuel testing equipment, including a handle, a first compartment configured to securely hold a fuel testing jar, and a second compartment configured to securely hold a fuel cap, wherein the first and second compartment openings are oriented toward the top of the caddy. Aspects of the present invention may also include a method of pre-flight fuel testing of an aircraft, including the steps of removing a fuel cap from the aircraft fuel tank and stowing the fuel cap from the aircraft fuel tank in a preflight fuel testing caddy, and removing a fuel testing jar from the preflight fuel testing caddy for fuel testing.

### CROSS-REFERENCE TO PRIOR APPLICATION

The present application claims the benefit of U.S. Provisional Application No. 63/607,824 filed Dec. 8, 2023, the entirety of which is incorporated by reference.

### TECHNICAL FIELD

The present disclosure relates to portable pre-flight caddies that can be used to directly assist in pre-flight fuel testing and other checks prior to aircraft takeoff, as well as indirectly assisting pre-flight testing by securely holding and dispensing tools for use in the same. Other aspects of the present disclosure include methods of using and manufacturing pre-flight caddies, as described more thoroughly below.

### BACKGROUND OF THE INVENTION

Aspects and embodiments of the present disclosure are portable pre-flight caddies that have been designed to allow pilots to more safely and efficiently pre-flight check their aircraft, along with methods of using and manufacturing the same. Aspects and embodiments of the present disclosure include a built-in funnel designed to prevent tested fuel from spilling on aviators' hands when replacing back into the fuel tank(s), as aviation gas (AvGas) is considered to be potentially carcinogenic. Additionally, caddies of the present disclosure keep all of the fuel testing supplies together so as to prevent dirt, grime, oil, dew, etc. from contaminating the pre-flighting materials.

The caddy is designed to hold a GATS (Gasoline Analysis Test Separator) fuel sump jar, a fuel dipstick, and the fuel cap. Together, these items can be used to pre-flight check the airplane. The funnel built into the caddy fits into the fuel filler hole in the wing to ensure no fuel is spilled after checking the fuel sump sample. The caddy is designed, in some embodiments, to be able to be securely stowed during flight and during testing procedures as well, which may have to be done in inclement weather conditions.

### BRIEF SUMMARY OF THE INVENTION

Some embodiments of the present disclosure may include a caddy for securely storing aircraft pre-flight fuel testing equipment, including a handle, a first compartment configured to securely hold a fuel testing jar, and a second compartment configured to securely hold a fuel cap, wherein the first and second compartment openings are oriented toward the top of the caddy.

Further embodiments include a third compartment configured to securely hold a fuel dipstick. Still others include one or more exterior loops configured to securely hold a fuel dipstick.

Others include an interior funnel for returning tested fuel back into an aircraft fuel tank, wherein the funnel extends from the top to the bottom of the caddy, with the larger end of the interior funnel located at the top of the caddy and the exit spout of the funnel at the bottom of the caddy. In other embodiments of the present disclosure, the exit spout of the funnel is located within a removed portion in the bottom of the caddy, the exit spout and removed portion configured to hold the caddy securely attached to an opening of an aircraft fuel tank while in use.

In others, the exit spout of the funnel protrudes from the removed portion in the bottom of the caddy, such that the exit spout extends inside an opening of a fuel tank while in use. In yet other embodiments, the top of the internal funnel is located within the first or second compartments. In others, the top of the funnel is located within the first compartment.

Other embodiments further include an opening between the first and second compartments configured to drain fuel from the second compartment to the funnel located in the first compartment. In other embodiments, the handle extends over the top of the caddy; in others, the handle extends over the top and one side of the caddy.

In yet other embodiments, the first and second compartments are configured so that the fuel testing jar and fuel cap protrude above the top surface of the caddy during storage for easier removal.

In others, the caddy comprises solvent-resistant plastic.

Aspects of the present disclosure include a method of pre-flight fuel testing of an aircraft, including the steps of removing a fuel cap from the aircraft fuel tank; stowing the fuel cap from the aircraft fuel tank in a preflight fuel testing caddy, wherein the preflight fuel testing caddy includes a handle, a first compartment, securely holding a fuel testing jar, and a second compartment configured to securely hold the fuel cap, wherein the first and second compartment openings are oriented toward the top of the preflight fuel testing caddy, and removing the fuel testing jar from the preflight fuel testing caddy for fuel testing.

In other aspects, the preflight fuel testing caddy further comprises a third compartment or one or more exterior loops holding a fuel dipstick, and includes removing the fuel dipstick from the preflight fuel testing caddy to test the fuel in the aircraft fuel tank.

In yet other aspects, the preflight fuel testing caddy further comprises an interior funnel for returning tested fuel back into an aircraft fuel tank, wherein the funnel extends from the top to the bottom of the preflight fuel testing caddy, with the larger end of the interior funnel located at the top of the preflight fuel testing caddy and the exit spout of the funnel at the bottom of the preflight fuel testing caddy, further including: positioning the exit spout over the opening of the aircraft fuel tank; and dispensing tested, safe fuel from the GATS jar back into the fuel tank through the funnel.

In other aspects, the exit spout of the funnel protrudes from the removed portion in the bottom of the preflight fuel testing caddy, such that the exit spout extends inside an opening of a fuel tank while being positioned over the opening of the aircraft fuel tank. In others, the top of the funnel is located within the first compartment. In yet others, the preflight fuel testing caddy further includes an opening between the first and second compartments configured to drain fuel from the second compartment to the funnel located in the first compartment. In others, the handle extends over the top of the preflight fuel testing caddy.

### BRIEF DESCRIPTION OF THE DRAWINGS

The present invention(s) are illustrated by way of example and not limitation with reference to the accompanying drawings, in which like references generally indicate similar elements or features.

FIG. 1 depicts a right-back-top isometric front view of an example pre-flight caddy embodiment of the present disclosure;

FIG. 2 depicts a left-front-top isometric front view of an example pre-flight caddy embodiment of the present disclosure;

FIG. 3 depicts a right-front-top isometric view of an example pre-flight caddy embodiment of the present disclosure;

FIG. 4 depicts a left-back-top isometric view of an example pre-flight caddy embodiment of the present disclosure;

FIG. 5 depicts a top view of an example pre-flight caddy embodiment of the present disclosure;

FIG. 6 depicts a right-front-bottom isometric view of an example pre-flight caddy embodiment of the present disclosure;

FIG. 7 depicts an isometric view of an example two-compartment plus funnel pre-flight caddy embodiment of the present disclosure;

FIG. 8 depicts a bottom view of an example embodiment of the present disclosure;

FIG. 9 depicts a top view of an example two-compartment plus funnel plus fuel dipstick holder embodiment of the present disclosure;

FIG. 10 depicts an isometric view of an example two-compartment plus funnel and fuel dipstick holder embodiment of the present disclosure;

FIG. 11 depicts a top view of an example two-compartment plus funnel plus fuel dipstick holder embodiment of the present disclosure;

FIG. 12 depicts a top view of an example two-compartment plus fuel dipstick holder embodiment of the present disclosure.

FIG. 13 depicts a top view of an example two-compartment plus fuel dipstick holder embodiment of the present disclosure.

### DETAILED DESCRIPTION

Pilots, especially pilots of smaller consumer aircraft, such as Cessnas and the like, are trained to check the type and quality of fuel in the aircraft prior to flight. Contaminated fuel or the wrong type of fuel can cause significant problems during takeoff and flight. Testing of fuel usually involves placing fuel into a GATS Jar (Gasoline Analysis Test Separator), which can then be replaced back into the fuel tank after analysis (if clean), reducing unnecessary environmental contamination from disposal of the tested fuel.

Pilots thus should keep a GATS jar and associated items such as a fuel dipstick, integrated fuel cap and checklist book within the plane. These are commonly stowed along with other items such as windshield cleaner, oil, cowl plugs, rags, loosely in an open plastic tub or something similar in the cockpit or other stowage area.

The fuel testing procedure commonly involves the GATS fuel sump jar and fuel dipstick being removed from the tub and rested on the wing next to the fuel cap. The fuel cap is removed first, and the fuel dipstick is inserted to provide a measurement of how much fuel is left in the tank. Once it is confirmed that there is a sufficient fuel supply, the GATS fuel sump jar is used to sump fuel from each sump point underneath the aircraft. The GATS jar is then checked to verify the fuel is not contaminated before pouring the fuel back into the fuel tank. At that point, the fuel cap is replaced in the wing and the process is repeated on the other wing.

Holding all these testing items can be challenging while moving around the aircraft, especially during cold weather months. Additionally, these items must be placed on the wing when not in use and can be blown off during windy days; wind, pilot error, or other events can also cause aviation fuel to spill onto pilot hands when pouring fuel from the GATS jar. Aviation gas is a skin irritant and carcinogenic, which should not come into contact with human skin per the manufacturers' recommendations. Thus, gloves are recommended when dispensing fuel, which is another potential item to be stowed.

The present disclosure describes pre-flight caddies and methods of manufacture and use designed to improve the safety and efficacy of the pre-flight testing process. Further, the small footprint, stable design, and tight hold of testing items in some embodiments aids in secure placement and stowage before, during and after testing, especially in inclement weather conditions. In some aspects and embodiments, the caddy can directly serve as a funnel to aid in placing fuel back into the fuel tank after testing.

For purposes of description, “top” and “bottom” in the context of this application, refers to the orientation of the caddy when in normal use. For instance, the funnel portion of the caddy, in normal use, would be orientated so that the narrow end of the funnel points toward the bottom of the caddy. For definitional purposes, the handle per se is not considered to be the “top surface” of the caddy: in some aspects and embodiments, a handle may extend or be extendable beyond the top surface of the caddy to aid in transport of the caddy. Note that in some aspects and embodiments, the handle can retract or be placed into a depression in the top surface and thus be substantially contiguous or comprise part of the top surface as needed to reduce the profile of the caddy in storage or for other uses, as described more fully below. Similarly, for definitional purposes, the “front” of the caddy is, where one is present, the narrower side of the caddy closest to the funnel; “back”, “right” and “left” are orientated based on the location of the “front”, with the “left” and “right” sides being located on the wider sides of the caddy.

Upper compartments. In some aspects and embodiments, the caddy has one or more compartments designed to hold a fuel cap (i.e., from an airplane gas tank) and GATS jar during stowage or fuel testing, as needed. In some embodiments, the compartments are open toward the top of the caddy to allow the compartments to hold items more easily, but other orientations are possible. In some aspects and embodiments, the openings of the compartments are substantially square in outline when viewed from the outside of the caddy. In some embodiments, the compartments have a non-square shape when viewed from outside of the caddy, such as, e.g., a circle or rounded square or rectangle. In some embodiments and aspects, the compartments and compartment openings are shaped to conform with the standard sizes of GATS jars and/or fuel caps, and/or other components that may be stored in the caddy if desired. For example, some embodiments may contain additional compartments to store a GATS jar, a fuel cap, and additional compartment(s) to contain other materials such as rags, gloves and the like.

In other aspects and embodiments, the depth of the compartments may be varied to allow stored components such as GATS jars and fuel caps to sit below the upper surface of the caddy, substantially at the surface of the caddy, or above the surface of the caddy as needed for easy access and secure storage. Thus, in some aspects and embodiments, a stored fuel cap may sit substantially below the top surface of the caddy, substantially even with the top level of the caddy, or protrude to some extent above the surface of the caddy when placed in a compartment for storage and transport to allow for easy visibility of the cap. Similarly, in some aspects and embodiments, the top of a stored GATS jar may sit substantially at the top level of the caddy, below the top surface of the caddy, or protrude to some extent above the surface of the caddy when placed in a compartment for storage and transport.

In some aspects and embodiments, the top of the funnel (described more fully below) may be configured to accept a GATS jar, so that the funnel may be used to dispense fuel into the gas tank when the GATS jar is removed, and store the GATS jar when not in use.

Aspects and embodiments may have one or more upper compartments, two or more upper compartments, or three or more upper compartments, in some embodiments each designed to hold one or more fuel testing components. In some embodiments and aspects, upper compartments and/or the funnel opening, may be in communication with each other such as with holes or removed areas of separation walls, to facilitate storage, stowed testing items removal, or provide room for protruding parts of testing items, such as the metal rod on some GATS jars.

Other compartments and holders. In some aspects and embodiments, one or more horizontal and/or vertical or angled slots may be placed on the top or sides of the caddy to accept, store and transport a fuel dipstick or other devices. In some aspects or embodiments, the slot contains one or more detents to hold the dipstick or other equipment securely in the slot when not in use. The slot may extend the full extent of the caddy (i.e. be open on both ends) or be closed on one end (in some embodiments, the bottom-most end) to prevent fuel spillage from remnant fuel on the dipstick.

In some aspects and embodiments, the caddy may comprise one or more rows of one or more exterior loops designed to hold a fuel dipstick in place. In some aspects and embodiments, a fuel dipstick may be slid through a row comprising one or more exterior loops on the sides or the top of caddy to store the fuel dipstick. The loops may be arranged in a line at an angle, horizontally or vertically, in order to sequentially accept a portion of the dipstick or other item as it is slid into the loops. The loops may define holes that are differentially sized for each other and/or define a hole that is open at both ends or be closed at one end or the other, to secure the fuel dipstick or other item securely. The loops may be made of the same material as the rest of the caddy, or be a different material. In some embodiments, the loops may comprise a row of one or more straps which may be tightenable, and secured to the rest of the body of the caddy by various means known in the art.

In some aspects and embodiments, the slot to accept a dipstick or other item may be vertical. In some aspects and embodiments, the slot may be in line with the sides of the handle. In some aspects and embodiments, a series of loops in vertical alignment with each other may be used to accept a fuel dipstick or other item. In some aspects, the bottom-most loop may be closed to hold the dipstick or other item in place vertically during storage and/or transport.

Funnel. In some aspects and embodiments, the pre-flight caddy includes an internal funnel to aid fuel or other liquids to be dispensed into a fuel tank or other container. In some embodiments, the top of the internal funnel extends to the top surface of the caddy and the opening of the funnel is substantially rectangular. In some embodiments, the funnel has a non-rectangular shape when viewed from the outside of the caddy, such as, e.g., a circle, rounded rectangle or square, or oval.

In some aspects and embodiments, the funnel extends through the entirety of the caddy body, such that the exit spout of the funnel has an opening in the bottom of the caddy. In general, the exit spout of the funnel is configured to fit within, around and/or over or on top of the opening of a fuel tank, in order to avoid spillage of fuel. In some embodiments and aspects, the bottom of the caddy has a portion removed around the exit spout such to aid in fuel being dispensed only into the fuel tank. In some aspects and embodiments, the opening extends around the exit spot such that the fuel spout fits into the fuel tank opening and the caddy opening engages the fuel tank opening, within and/or around the fuel tank opening, to better secure the funnel when it use and/or the prevent fuel spillage.

In some aspects, the openings of the compartments and the top opening of the funnel(s) are separated by one or more (depending on the number of compartments and funnels present in an embodiment) separation areas on the body and/or surface of the caddy on the top surface of the caddy. A separation area separates adjacent compartments from each other, or a funnel from another funnel and/or compartment.

As noted above, the funnel may itself be used as a storage component, storing, for example a GATS jar when not in use to dispense fuel.

In some embodiments and aspects, upper compartments, such as may be in communication with each other, such as with holes or removed areas of separation walls, for example, to facilitate drainage of fuel remnants from the fuel cap into the funnel, and thus back into the gas tank, when the fuel cap is stowed after being removed from the fuel tank.

Handle In some aspects and embodiments, the caddy has a handle, designed to enable easy movement of the caddy and its contents from place to place when in use. In some embodiments, the handle extends substantially solely over one or more of the separation areas when viewed in top view, i.e., not substantially extending over the openings of the funnel and/or compartments. This allows, in some embodiments and aspects, fluids to easily dispensed into the funnel and room for objects such as the GATS jar and fuel cap to easily stored and removed from the compartments and be visible while in storage. In some embodiments, the handle may extend over one, two or three separation areas or more than one separation area. Thus, a handle may extend in one substantially straight length, when viewed from above, over the top surface of the caddy, or may branch, wherein the branches are in some embodiments at right angles from each other.

In some aspects and embodiments, the handle may extend above the top surface for a sufficient distance to allow for one hand of a user to easily hold the caddy above the top surface and any or some of the stored materials. In some aspects and embodiments, the handle extends a sufficient distance to allow a gloved hand (with gloves of various thicknesses such as protective gloves, work gloves, winter gloves of various thicknesses) of a user to easily hold the caddy above the top surface and any or some of the stored materials. In some aspects and embodiments, the handle extends a sufficient distance to allow a winter-gloved hand of a user to easily hold the caddy above the top surface and any or some of the stored materials.

In some aspects, the handle is removable from the caddy. In some aspects and embodiments, the handle may be slidable up and down vertical slots present in the sides of the caddy to reduce the profile of the caddy when not in use. In some aspects and embodiments, the handle may swivel or fold down over a side of the caddy to reduce the profile of the caddy when not in use; the handle may in some embodiments swivel or fold down into a depression/indent present on the top or side(s), or at the junction of the top and side(s) when not in use. Some aspects and embodiments may use locking mechanisms such a detent or locking hinge to more securely hold the handle in a storage (i.e. down) and/or transport position (i.e. upright).

In some aspects or embodiments, the handle may extend over the top and also over one or more sides of the caddy for more versatility for moving the caddy.

In some aspects and embodiments, indentations may be present in the bottom, top or sides to reduce the overall weight of the caddy. In some aspects and embodiments, the intended use of each compartment may be indicated with labels.

In some aspects or embodiments, the caddy may be constructed from durable, solvent-resistant molded or 3D-printed plastic, or other suitable materials or methods known in the art. Embodiments may be constructed out of various materials, such as metal, wood etc. or a combination of materials. Various techniques may be use to increase grip friction, for example on the handle or bottom of the caddy, including rubber pads, grooved surfaces

Methods of use of the pre-flight caddy include one of more of the following: removing a fuel cap from an airplane and stowing it in embodiments of the fuel caddy as described above; removing a stowed GATS jar from the caddy for fuel testing; removing a fuel dipstick from the caddy for testing; placing the funnel exit spout over the opening of the aircraft fuel tank, pouring tested, safe fuel from the GATS jar back into the fuel tank through the funnel; replacing the stowed fuel cap back on the fuel tank; and restowing the GATS jar and fuel dipstick.

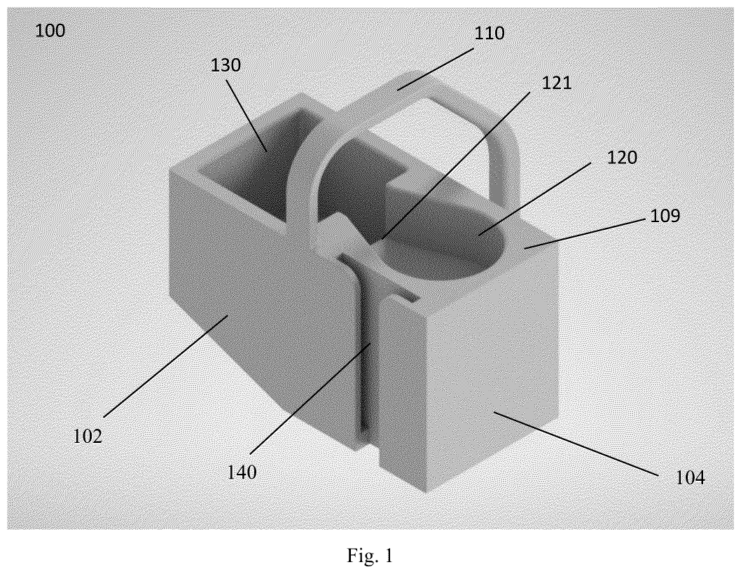

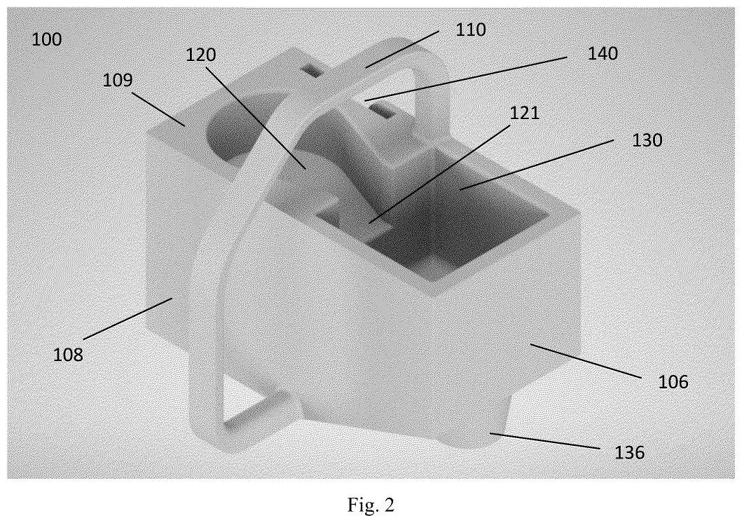

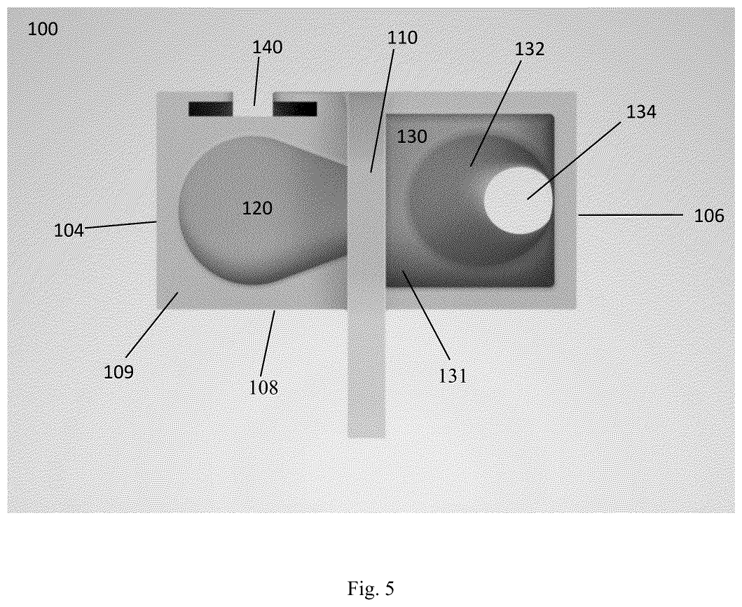

FIGS. 1-6 show various views of an example embodiment 100 of the present disclosure. Referring to FIG. 1, a right-back-top isometric front view of example embodiment 100 has a right side 102, back and top 109 visible. Handle 110 extends over the top 106 of the caddy and over the left side 108 (see FIG. 2, a left-front-top isometric front view of example embodiment 100). No particular left-right orientation of the device is required; in some embodiments, the position of handle 110 and slot 140 extending from top 109 to configured to hold a fuel dipstick may run in the opposite orientation or occur on opposite sides. Fuel dipstick holder 140 is located opposite side 108 to allow for unobstructed use of handle 110, but other orientations are possible in some embodiments. Opening 120 is located on top 109 and is designed to hold an aircraft fuel cap, with channel 121 allowing excess fuel to flow from the fuel cap into opening 130 and then into funnel 132 (see, e.g. FIG. 5). Opening 130 is configured to accepts GATS jar, but may be modified in other embodiments to hold fuel testing jars of other sizes.

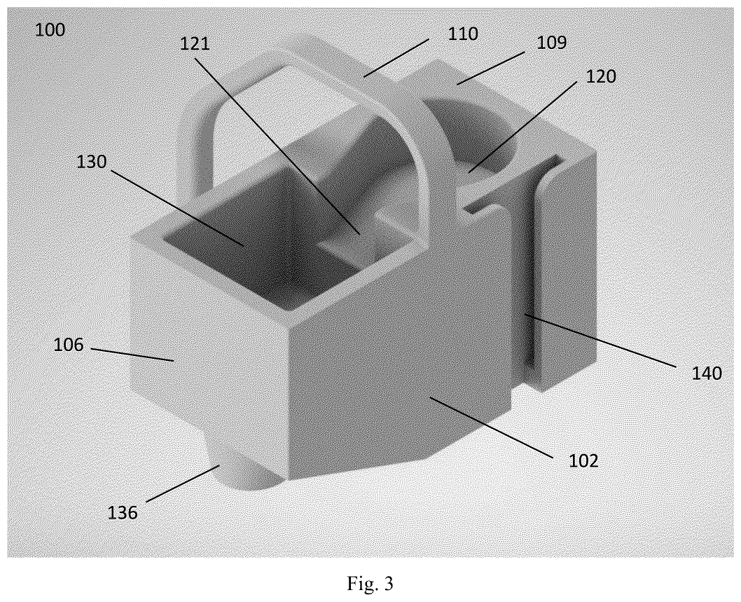

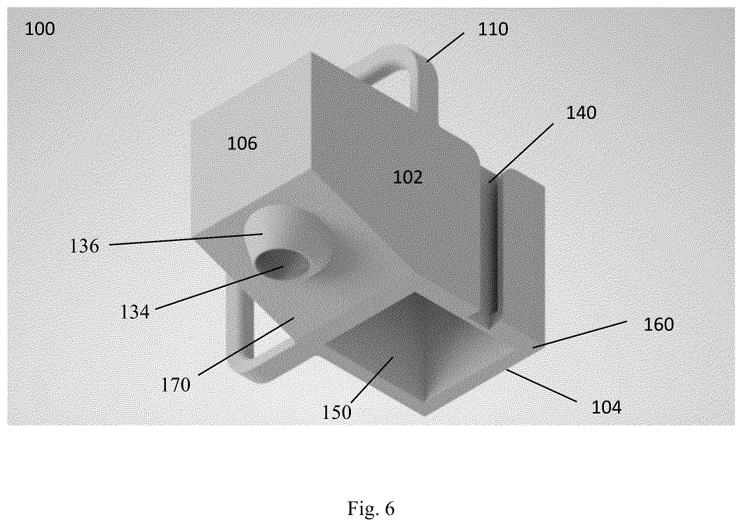

FIG. 3 is a right-front-top isometric view of the example caddy 100, showing the front 106 of the fuel caddy and the orientation of funnel spout 136, protruding from the bottom 160 (see e.g. FIG. 6) of the example caddy.

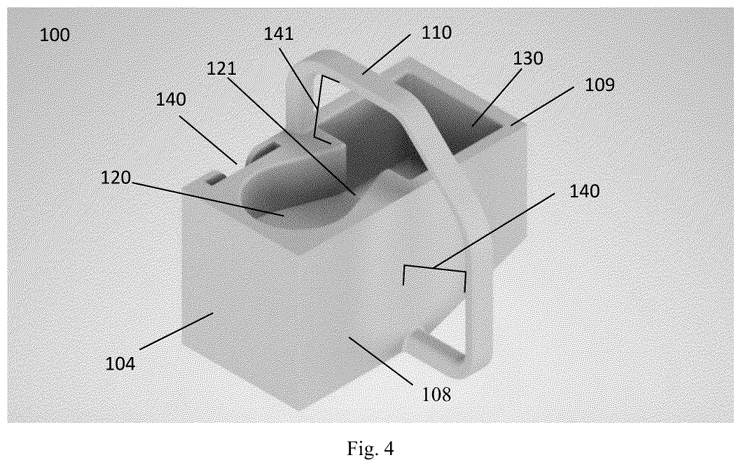

FIG. 4 is a left-back-top isometric view of the example caddy 100. Shown here are distances 140 and 141, which indicate the distances between handle 110 and side 108 and top 109, respectively. In some embodiments, these distances are sufficient to allow for the caddy to be carried comfortably in one of two orientations with one or more hands wearing winter gloves, as needed.

FIG. 5 is a top view of the example embodiment 100, showing the orientation of handle 110, in some embodiments, between openings 120 and 130, to allow items to be easily placed in and taken out of the openings. Opening 130 has a shelf 131, as funnel 132 is smaller in diameter than opening 130 and is recessed below the top 109 of the caddy. Shelf 132 is designed to hold a GATS jar above the funnel, allowing for the dual use of opening 130 for storage and fuel dispensing through built-in funnel 132.

FIG. 6 shows the bottom 160 of example embodiment 100. Funnel spout 136 is placed on upward-angled portion 170 of bottom 160 to allow for spout 136 to protrude from bottom 160 and caddy 100 still rest stably on a surface. Fuel is dispensed into a fuel tank through opening 134 of the funnel. In some embodiments, spout 136 is configured to fit inside a fuel tank opening during use to avoid fuel spillage. In further embodiments, caddy 100 may be tiltable onto upward-angled portion 170 to aid in fuel draining and dispensing from the caddy (for instance from opening 120 to 130 through channel 121). Opening 150 is extends into the body of caddy 100 and is designed to save weight and material costs in some embodiments.

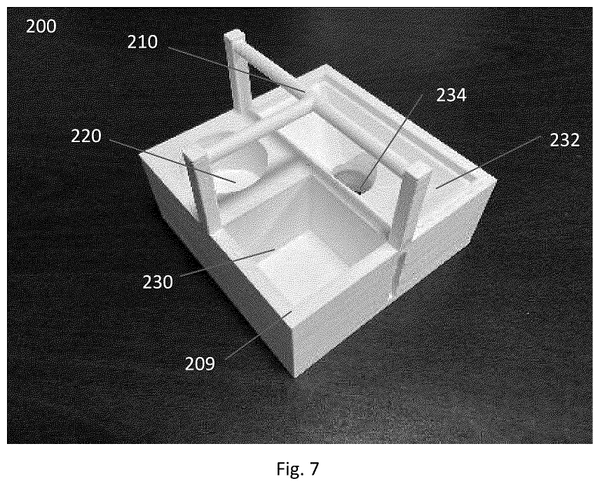

FIG. 7 shows another example embodiment 200 showing an opening 230 designed to hold a GATS jar, opening 220 designed to hold a fuel cap, and funnel 232 with opening 234 extending through the caddy to allow fuel to be dispensed. Handle 210 is in three parts, wherein the handle parts extend between the openings on the top 209 of caddy 200.

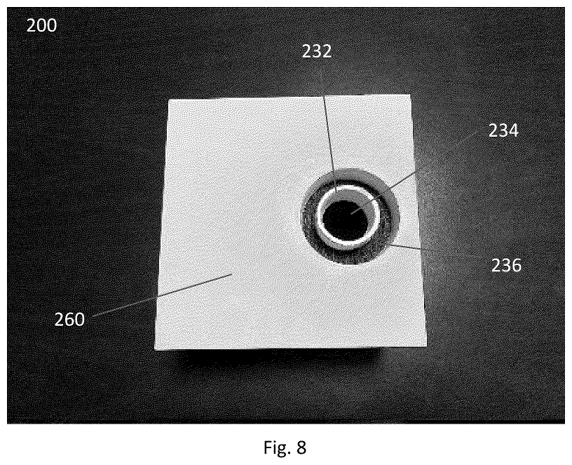

FIG. 8 shows a bottom view of an example embodiment 200, with another embodiment of a funnel spout 232 to minimize fuel loss. Spout 232 on bottom 260 of caddy 200 is configured to fit inside a fuel tank opening, placing opening 232 within the tank. To allow for the caddy to rest securely on bottom 260, fuel spout 232 is recessed in opening 236. Opening 236 may also be configured, in some embodiments, to fit over a fuel cap opening.

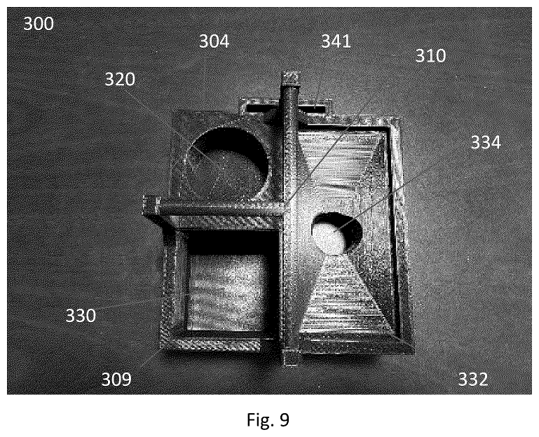

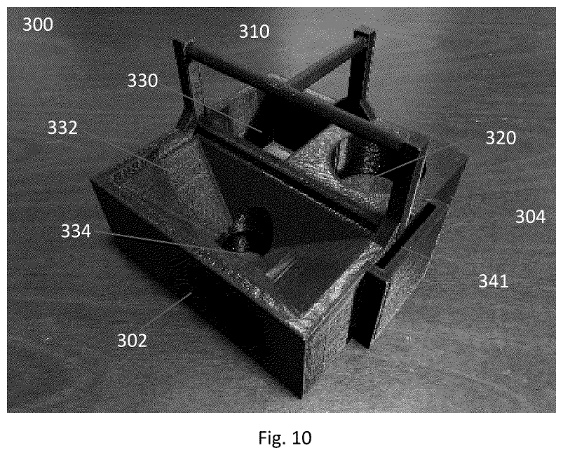

FIG. 9 shows a top view and FIG. 10 an isometric view of an example two-compartment plus funnel plus fuel dipstick holder embodiment 300 of the present disclosure. In this embodiment fuel dipstick holder is held to side 304 by loops 341.

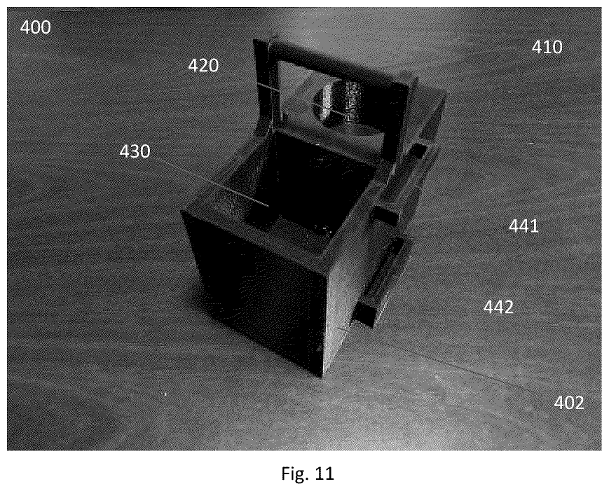

FIG. 11 shows an isometric view of an example two-compartment plus funnel plus fuel dipstick holder embodiment 500, where the fuel dipstick is held to the side of the caddy 400 by upper loop 441 and lower loop 442.

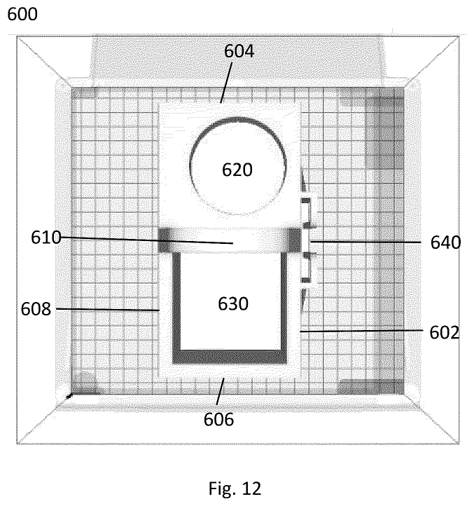

FIG. 12 shows a top view of a two-compartment plus fuel dipstick holder embodiment 600, with fuel cap holder opening 620 and GATS jar opening 630 in the top 609 of caddy 600. Fuel dipstick holder 640 protrudes from the side 602 of the caddy.

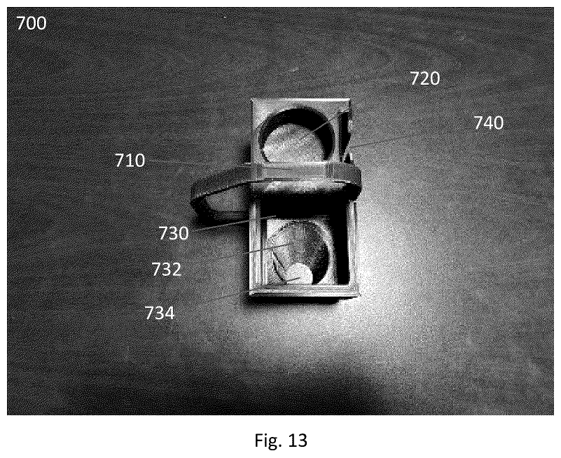

In FIG. 12, the fuel dipstick holder 640 is proximate and below handle 610, but can be placed in other locations as seen in the example caddy embodiment 700 in FIG. 13, where the fuel dipstick holder 740 is placed to the side of handle 710.

1. A caddy for securely storing aircraft pre-flight fuel testing equipment, comprising
a handle,
a first compartment configured to securely hold a fuel testing jar, and
a second compartment configured to securely hold a fuel cap,
wherein the first and second compartment openings are oriented toward the top of the caddy.

2. The caddy of claim 1, further comprising a third compartment configured to securely hold a fuel dipstick.

3. The caddy of claim 1, further comprising one or more exterior loops configured to securely hold a fuel dipstick.

4. The caddy of claim 1, further comprising an interior funnel for returning tested fuel back into an aircraft fuel tank, wherein the funnel extends from the top to the bottom of the caddy, with the larger end of the interior funnel located at the top of the caddy and the exit spout of the funnel at the bottom of the caddy.

5. The caddy of claim 1, wherein the exit spout of the funnel is located within a removed portion in the bottom of the caddy, the exit spout and removed portion configured to hold the caddy securely attached to an opening of an aircraft fuel tank while in use.

6. The caddy of claim 5, wherein the exit spout of the funnel protrudes from the removed portion in the bottom of the caddy, such that the exit spout extends inside an opening of a fuel tank while in use.

7. The caddy of claim 4, wherein the top of the internal funnel is located within the first or second compartments.

8. The caddy of claim 7, wherein the top of the funnel is located within the first compartment.

9. The caddy of claim 8, further comprising an opening between the first and second compartments configured to drain fuel from the second compartment to the funnel located in the first compartment.

10. The caddy of claim 1, wherein the handle extends over the top of the caddy.

11. The caddy of claim 1, wherein the handle extends over the top and one side of the caddy.

12. The caddy of claim 1, wherein the first and second compartments are configured so that the fuel testing jar and fuel cap protrude above the top surface of the caddy during storage for easier removal.

13. The caddy of claim 1, wherein the caddy comprises solvent-resistant plastic.

14. A method of pre-flight fuel testing of an aircraft, comprising:
removing a fuel cap from the aircraft fuel tank;
stowing the fuel cap from the aircraft fuel tank in a preflight fuel testing caddy,
wherein the preflight fuel testing caddy comprises
a handle,
a first compartment, securely holding a fuel testing jar, and
a second compartment configured to securely hold the fuel cap,
wherein the first and second compartment openings are oriented toward the top of the preflight fuel testing caddy, and
removing the fuel testing jar from the preflight fuel testing caddy for fuel testing.

15. The method of claim 14, wherein the preflight fuel testing caddy further comprises a third compartment or one or more exterior loops holding a fuel dipstick, the method further comprising:
removing the fuel dipstick from the preflight fuel testing caddy to test the fuel in the aircraft fuel tank.

16. The method of claim 14, wherein the preflight fuel testing caddy further comprises an interior funnel for returning tested fuel back into an aircraft fuel tank, wherein the funnel extends from the top to the bottom of the preflight fuel testing caddy, with the larger end of the interior funnel located at the top of the preflight fuel testing caddy and the exit spout of the funnel at the bottom of the preflight fuel testing caddy, the method further comprising:
positioning the exit spout over the opening of the aircraft fuel tank; and
dispensing tested, safe fuel from the fuel testing jar back into the fuel tank through the funnel.

17. The method of claim 16, wherein the exit spout of the funnel protrudes from the removed portion in the bottom of the preflight fuel testing caddy, such that the exit spout extends inside an opening of a fuel tank while being positioned over the opening of the aircraft fuel tank.

18. The method of claim 16, wherein the top of the funnel is located within the first compartment.

19. The method of claim 16, wherein the preflight fuel testing caddy further comprises an opening between the first and second compartments configured to drain fuel from the second compartment to the funnel located in the first compartment.

20. The method of claim 14, wherein the handle extends over the top of the preflight fuel testing caddy.
